(19) United States
(12) Patent Application Publication
(10) Pub. No.: US 2012/0264975 A1
(43) Pub. Date: Oct. 18, 2012

(54) Phase Transfer Catalyst for Producing Aromatic Aldehyde Compound

(75) Inventors: Yu-Chin Lee, Taipei (TW); Hsien-Yu Chang, Chiayi City (TW); Han-Hsu Chen, Taichung City (TW); Yu-Sen Hou, Kaohsiung City (TW); Rong-Yi Liao, New Taipei City (TW)

(73) Assignee: UFC Corporation, Taipei (TW)

(21) Appl. No.: 13/086,470

(22) Filed: Apr. 14, 2011

(51) Int. Cl. C07C 211/63

(52) U.S. Cl. 564/282

(57) ABSTRACT

Provided is a phase transfer catalyst for producing an aromatic aldehyde compound, comprising a compound of formula I, wherein R₁ is hydrogen, methyl, ethyl, isopropyl, isobutyl or tertbutyl; R₄, R₅ and R₆ are independently selected from alkyl groups containing 1 to 6 carbon atoms; and X is fluorine, chlorine, bromine or iodine.

### BACKGROUND OF THE INVENTION

1. Field of the Invention

The present invention relates to a phase transfer catalyst (PTC) for synthesizing an aromatic aldehyde compound, which has simplified steps and is useful for obtaining a higher product yield in the process for producing synthetic aromatic aldehydes.

2. Description of the Prior Arts

Certain aromatic aldehyde compounds are used as fragrance, commonly named aldehyde fragrance. For example, lysmeral is one of the aromatic aldehyde compounds, whose production was firstly industrialized by Givaudan Co. and widely used in perfume and fragrance industries for producing soaps, detergents, cosmetics and other articles for daily use. Domestic consumption in some countries relies heavily on import from other countries and the demands for aldehyde fragrance, such as lysmeral, etc., are increasing. However, the worldwide market shares and patents for lysmeral are dominantly held by certain companies. Further, several disadvantages of industrial production for aromatic aldehyde compounds remain.

Current processes for industrial production of aldehyde fragrance have complicated steps and a low yield of less than 30%. In current processes for synthesizing aldehyde fragrance, production of alkyl-substituted benzaldehyde is critical. The last step of the current process is hydrogen-reduction, which is performed with palladium on carbon as catalyst. However, palladium, a noble metal, is expensive and increases total cost of the process. Patent NO. WO2007045641 owned by Badische Anilin- and Soda-Fabrik Corp. (BASF) aims at improving the final step for hydrogen-reduction of the above process. However, the reaction pressure is so high as 30 Bar, leading to difficulties in its industrialization.

To overcome the shortcomings, the present invention provides a process for producing an aromatic aldehyde compound to mitigate or obviate the aforementioned problems.

### SUMMARY OF THE INVENTION

Given the current techniques' lack of means for producing aromatic aldehyde compounds in an effective and economic way, the subject of the present invention is to provide a catalyst for producing an aromatic aldehyde compound, by which an aromatic aldehyde compound can be obtained without using high pressure-reduction equipments. The process for producing an aromatic aldehyde compound in accordance with the present invention has simplified steps and can help obtain a high product yield, so that it is suitable for industrial production of the aromatic aldehyde compounds. Accordingly, the present invention provides a phase transfer catalyst for producing an aromatic aldehyde compound, which comprises a compound of formula I:

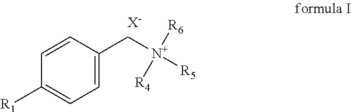

wherein R₁ is hydrogen, methyl, ethyl, isopropyl, isobutyl or tertbutyl; R₄, R₅ and R₆ are independently selected from alkyl groups containing 1 to 6 carbon atoms; and X is fluorine, chlorine, bromine or iodine.

Preferably, the phase transfer catalyst for producing an aromatic aldehyde compound in accordance with the present invention is adapted to a process for producing an aromatic aldehyde compound, wherein the process for producing an aromatic aldehyde compound comprises steps of:

converting alkyl-substituted or non-substituted benzene into a compound of formula II by halomethylation to form a compound of the following formula II;

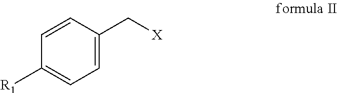

reacting the compound of formula II with alkyl aldehyde to react in presence of phase transfer catalyst at an appropriate reaction temperature under alkaline condition to obtain an aromatic aldehyde compound.

More preferably, the aromatic aldehyde compound as described above is lysmeral, floralozone, 2,2-dimethyl-3-(3-methylphenyl) propanal, 2-methyl-3-(p-isopropylphenyl) propanal, dimethyl-4-(1-methylethyl)benzenepropanal or 2,2-dimethyl-3-phenylpropanal.

Yet more preferably, the compound of formula I as described above is 4-tertbutylbenzyl trimethyl ammonium bromide, 4-tertbutylbenzyl triethyl ammonium bromide, or 4-tertbutylbenzyl triethyl ammonium iodide.

Based on the previous description, the phase transfer catalyst for producing an aromatic aldehyde compound in accordance with the present invention has the following advantages: (1) raw material for producing the phase transfer catalyst of the present invention is easy to be obtained and economic; (2) use of the phase transfer catalyst of the present invention can avoid use of hydrogen-reduction under high pressure; (3) the phase transfer catalyst of the present invention helps reaction catalyzed by the same to have a yield over 60% and content of target compound in the final product is over 95%, demonstrating that the phase transfer catalyst of the present invention is useful for industrial production of aromatic aldehyde compounds.

Other objectives, advantages and novel features of the invention will become more apparent from the following detailed description when taken in conjunction with the accompanying drawings.

### DETAILED DESCRIPTION OF THE PREFERRED EMBODIMENTS

The present invention uses an aromatic phase transfer catalyst as catalyst in a process for producing an aromatic aldehyde compound, which comprises a compound of formula I:

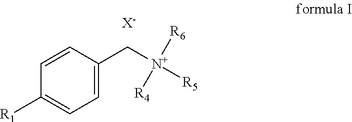

wherein R₁ is hydrogen, methyl, ethyl, isopropyl, isobutyl or tert-butyl; R₄, R₅, R₆ are independently alkyl containing 1 to 6 carbon atoms, and X is fluorine, chlorine, bromine or iodine.

The aromatic type phase transfer catalyst for producing an aromatic aldehyde compound in accordance with the present invention can be applied in Route A as follows:

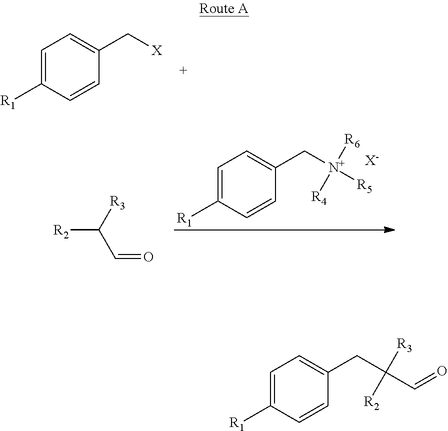

wherein R₂ and R₃ are independently hydrogen or methyl group.

The aromatic type phase transfer catalyst for producing an aromatic aldehyde compound is derived from the same raw materials as the process for producing an aromatic aldehyde compound. The process for producing an aromatic aldehyde compound with the aromatic phase transfer catalyst has a higher yield than that of the process with general alkyl type phase transfer catalyst. According to the following examples, reaction catalyzed by the phase transfer catalyst for producing an aromatic aldehyde compound in accordance with the present invention has advantages of requiring a moderate reaction condition, being easy to be handled, having high product yield (overall yield >60%), not causing corrosion of equipments, not requiring reduction or hydrogenation under high pressure, having purity over 95% and being suitable for industrial production.

The present invention was further illustrated by the following examples; it should be understood that the examples and embodiments described herein are for illustrative purposes only and should not be construed as limiting the embodiments set forth herein.

### EXAMPLES

### Example-1

### Preparation of 4-tertbutylbenzyl trimethyl ammonium bromide

The present example was performed by following scheme I and synthesis procedure as described below:

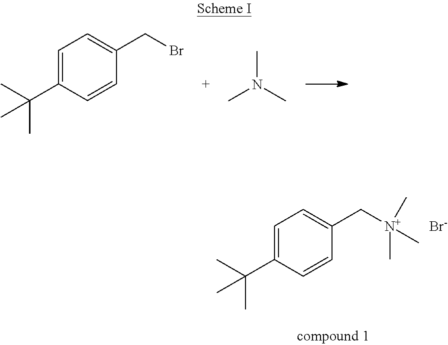

50 grams (220.1 mmol) of 4-tertbutylbenzyl bromide was dissolved in 200 mL anhydrous ethanol and 14.31 grams (242.1 mmol) of trimethyl amine was added, followed by being refluxed for 2 hours. Reaction solution was left standing overnight and filtered to remove liquid and obtain a solid. The solid was washed with anhydrous ethanol for three times and dried in vacuum at 25° C. The compound 1, 74 grams, was obtained with a yield of 83.7%.

### Example-2

### Preparation of Phase Transfer Catalyst 4-tertbutylbenzyl triethyl ammonium bromide (Route 1)

The present example was performed by following scheme II and synthesis procedure as described below:

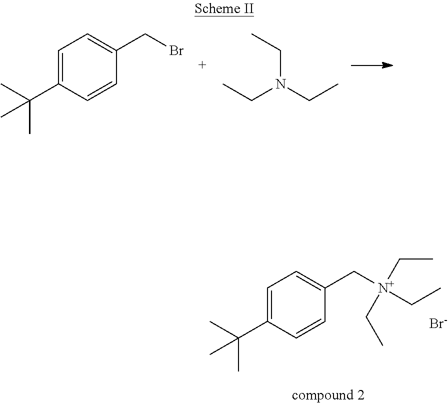

50 grams (220.1 mmol) of 4-tertbutylbenzyl bromide was dissolved in 200 mL anhydrous ethanol and 24.5 grams (242.1 mmole) of triethyl amine was added, followed by being refluxed for 2 hours. Reaction solution was left standing overnight and filtered to remove liquid and obtain a solid. The solid was washed with anhydrous ethanol for three times and dried in vacuum at 25° C. The compound 2, 51.89 grams, was obtained with a yield of 71.8%.

### Example-3

### Preparation of Phase Transfer Catalyst 4-tertbutylbenzyl triethyl ammonium bromide (Route 2)

The present example was performed by following scheme III and synthesis procedure as described below:

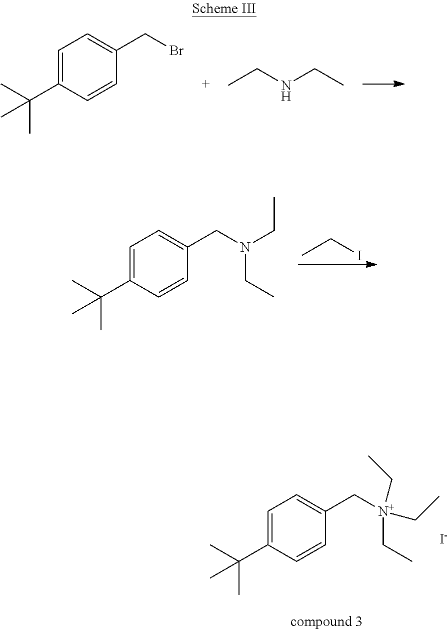

50 grams (251.2 mmol) of 4-tertbutylbenzyl bromide was dissolved in 200 mL anhydrous ethanol, followed by purging with nitrogen for three times. 17.7 grams (242.1 mmol) of triethyl amine was added into the charging funnel and dropped slowly into reaction solution. The reaction solution was refluxed for 3 hours when reaction was completed and then subjected to dryness in vacuum (15 to 20 torrs) to remove solvent. The residue was distilled in vacuum to collect a desired product. The desired product was then dissolved in 300 mL tetrahydrofuran (THF) and stirred at room temperature to form a mixed solution. 171.67 grams (1.1 mol) of ethyl iodide was dropped into the mixed solution, and then the mixed solution was stirred at room temperature for another 3 hours after addition to form a solid and stood overnight and was filtered off solvent to obtain a solid product. The solid product was washed with anhydrous ethanol for three times and dried in vacuum at 25° C. The compound 3, 64.62 grams, was obtained with a yield of 78.2%.

### Comparative Example-1

### Preparation of Lysmeral with Alkyl Phase Transfer Catalyst

2.3 grams (57.7 mmol) of sodium hydroxide, 0.33 grams (0.88 mmol) of tetrabutyl ammonium iodide, 7.5 mL of water, 4.2 mL of toluene, 1 mL of THF were added into a flask and heated to 70 to 75° C. Mixture of 10 grams (44.0 mmol) of 4-tertbutylbenzyl bromide and 3.55 grams (61.2 mmol) of propanal was added dropwise into the flask within two hours while the mixed solution was vigorously stirred, followed by stirring at 70 to 75° C. for another 3 hours after addition was finished. Reaction was traced by gas chromatography. The reaction mixture was extracted with 30 mL of water to obtain an organic layer when reaction was completed. The organic layer was dehydrated with anhydrous sodium sulfate, filtered off salt to isolate mother liquid, and stripped off solvent by vacuum distillation. 4.84 grams of lysmeral was obtained with a yield of 53.8%.

### Example-4

### Preparation of Lysmeral with Aromatic Phase Transfer Catalyst

Compound 1, 4-tertbutylbenzyl trimethyl ammonium bromide, which was prepared in example 1, was used in the present example as aromatic type phase transfer catalyst (PTC). The present example was performed by following scheme IV and synthesis procedure as described below:

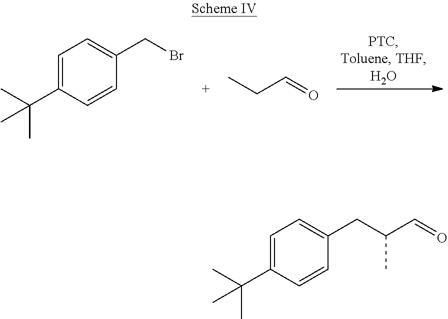

2.3 grams (57.7 mmol) of sodium hydroxide, 0.26 grams (0.88 mmol) of 4-tertbutylbenzyl trimethyl ammonium bromide, 7.5 mL of water, 4.2 mL of toluene, 1 mL of THF were added into a flask and heated to 70 to 75° C. Mixture of 10 grams (44.0 mmol) of 4-tertbutylbenzyl bromide and 3.55 grams (61.2 mmol) of propanal was added dropwise and slowly into the flask within 2 hours while the mixed solution in the flask was vigorously stirred, followed by stirring at 70 to 75° C. for another 3 hours after addition was finished. Reaction was traced by gas chromatography. The reaction mixture was extracted with 30 mL of water to obtain an organic layer when reaction was completed. The organic layer was dehydrated with anhydrous sodium sulfate, filtered off salt to isolate mother liquid, and stripped off solvent by vacuum distillation. 7.43 grams of lysmeral was obtained with a yield of 82.6%.

### Example-5

### Preparation of Lysmeral with Aromatic Phase Transfer Catalyst

Compound 2 prepared in example 2, i.e. 4-tertbutylbenzyl triethyl ammonium bromide, was used in the present example as PTC. The present example was performed by following scheme IV as described above and synthesis procedure as described below:

2.3 grams (57.7 mmol) of sodium hydroxide, 0.29 grams (0.88 mmol) of 4-tertbutylbenzyl triethyl ammonium bromide, 7.5 mL of water, 4.2 mL of toluene, 1 mL of THF were added into a flask and heated to 70 to 75° C. Mixture of 10 grams (44.0 mmol) of 4-tertbutylbenzyl bromide and 3.55 grams (61.2 mmol) of propanal was added dropwise and slowly into the flask within 2 hours while the mixed solution in the flask was vigorously stirred, followed by stirring at 70 to 75° C. for another 3 hours when addition was finished. Reaction was traced by gas chromatography. The reaction mixture was extracted with 30 mL of water to obtain an organic layer when reaction was completed. The organic layer was dehydrated with anhydrous sodium sulfate, filtered off salt to isolate mother liquid, and stripped of solvent by vacuum distillation. 7.47 grams of lysmeral was obtained with a yield of 83.1%.

### Example-6

### Preparation of Lysmeral with Aromatic Phase Transfer Catalyst

Compound 3 prepared in example 3, i.e. 4-tertbutylbenzyl triethyl ammonium iodide, was used in the present example as PTC. The present example was performed by following scheme IV as described above and synthesis procedure as described below:

2.3 grams (57.7 mmol) of sodium hydroxide, 0.33 grams (0.88 mmol) of 4-tertbutylbenzyl triethyl ammonium iodide, 7.5 mL of water, 4.2 mL of toluene, 1 mL of THF were added into a flask and heated to 70 to 75° C. Mixture of 10 grams (44.0 mmol) of 4-tertbutylbenzyl bromide and 3.55 grams (61.2 mmol) of propanal was added dropwise and slowly into the flask within 2 hours while the mixed solution in the flask was vigorously stirred, followed by stirring at 70 to 75° C. for another 3 hours when addition was finished. Reaction was traced by gas chromatography. The reaction mixture was extracted with 30 mL of water to obtain an organic layer when reaction was completed. The organic layer was dehydrated with anhydrous sodium sulfate, filtered off salt to isolate mother liquid, and stripped off solvent by vacuum distillation. 7.49 grams of lysmeral was obtained with a yield of 83.3%.

Table 1 showed the results of preparation of lysmeral according to the method as described in examples 4 to 6, demonstrating differences between yields of lysmeral under a condition of various catalysts and temperatures.

TABLE 1

| Reaction | | |
|---|---|---|
| temperature | phase transfer catalyst^{} | Yield |
| 20-25° C. | 4-tertbutylbenzyl trimethyl ammonium | no reaction |
| | bromide | |
| | 4-tertbutylbenzyl triethyl ammonium | no reaction |
| | bromide | |
| | 4-tertbutylbenzyl triethyl ammonium | no reaction |
| | iodide | |
| | tetrabutyl ammonium iodide | no reaction |
| 50-60° C. | 4-tertbutylbenzyl trimethyl ammonium | 63.1% |
| | bromide | |
| | 4-tertbutylbenzyl triethyl ammonium | 63.7% |
| | bromide | |
| | 4-tertbutylbenzyl triethyl ammonium | 63.8% |
| | iodide | |
| | tetrabutyl ammonium iodide | 50.9% |
| 70-75° C. | 4-tertbutylbenzyl trimethyl ammonium | 82.6% |
| | bromide | |
| | 4-tertbutylbenzyl triethyl ammonium | 83.1% |
| | bromide | |
| | 4-tertbutylbenzyl triethyl ammonium | 83.3% |
| | iodide | |
| | tetrabutyl ammonium iodide | 53.8% |

^{}0.02 equivalent of phase transfer catalyst was used herein.

As shown in Table 1, reactions utilizing 4-tertbutylbenzyl trimethyl ammonium bromide and 4-tertbutylbenzyl triethyl ammonium bromide as phase transfer catalyst have higher yields of aromatic aldehyde compounds than those utilizing tetrabutyl ammonium iodide.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A phase transfer catalyst for producing an aromatic aldehyde compound, comprising a compound of formula I: 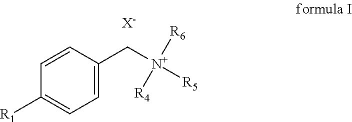
wherein R₁ is hydrogen, methyl, ethyl, isopropyl, isobutyl or tertbutyl; R₄, R₅ and R₆ are independently selected from alkyl groups containing 1 to 6 carbon atoms; and X is fluorine, chlorine, bromine or iodine.

2. The phase transfer catalyst for producing an aromatic aldehyde compound of claim 1, which is adapted to a process for producing an aromatic aldehyde compound, wherein the process for producing an aromatic aldehyde compound comprises steps of:
converting alkyl-substituted or non-substituted benzene into a compound of formula II by halomethylation to form a compound of the following formula II; 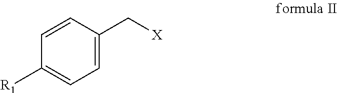
reacting the compound of formula II with alkyl aldehyde in presence of the phase transfer catalyst at an appropriate reaction temperature under alkaline condition to obtain an aromatic aldehyde compound.

3. The phase transfer catalyst for producing an aromatic aldehyde compound of claim 2, wherein the aromatic aldehyde compound is lysmeral, floralozone, 2,2-dimethyl-3-(3-methylphenyl)propanal, 2-methyl-3-(p-isopropylphenyl)propanal, dimethyl-4-(1-methylethyl)benzenepropanal or 2,2-dimethyl-3-phenylpropanal.

4. The phase transfer catalyst for producing an aromatic aldehyde compound of claim 1, wherein the compound of formula I is 4-tertbutylbenzyl trimethyl ammonium bromide, 4-tertbutylbenzyl triethyl ammonium bromide, or 4-tertbutylbenzyl triethyl ammonium iodide.
